(19) United States
(12) Patent Application Publication
(10) Pub. No.: US 2022/0409921 A1
(43) Pub. Date: Dec. 29, 2022

(54) TRANSCRANIAL STIMULATOR FOR REHABILITATION BASED ON PHOTOBIOMODULATION MECHANISM

(71) Applicant: WELLSCARE CO., LTD, Suwin-si (KR)

(72) Inventors: Sung Won LEE, Yongin-si (KR); Sanghyun CHANG, Suwon-si (KR); Yong Hyun CHO, Suwon-si (KR)

(21) Appl. No.: 17/903,638

(22) Filed: Sep. 6, 2022

Related U.S. Application Data
(63) Continuation-in-part of application No. 17/477,143, filed on Sep. 16, 2021.
Application No. 17/477,143 is a continuation of application No. 16/074,211, filed on Jul. 31, 2018, now Pat. No. 11,154,723.

(30) Foreign Application Priority Data
Nov. 22, 2016 (KR) 10-2016-0155918
May 15, 2017 (KR) 10-2017-0059923

(51) Int. Cl. A61N 5/06; A61N 5/067

(52) U.S. Cl. CPC A61N 5/0616; A61N 5/0619; A61N 2005/0645; A61N 2005/0651; A61N 5/067

(57) ABSTRACT

A transcranial stimulator includes a main body formed in a shape that covers part or all of a head surrounding a skull, a light emission module configured in the main body to transmit light energy to a brain, and a controller configured to control an operation of the light emission module, wherein the light emission module includes a first optical device emitting light in a wavelength band of 630 nm to 680 nm toward the skull and a second optical device emitting light in a wavelength band of 780 nm to 990 nm toward the skull.

### CROSS-REFERENCE TO RELATED APPLICATION

The present application is a Continuation-In-Part (CIP) application of U.S. patent application Ser. No. 17/477,143 filed on Sep. 16, 2021, in The United States Patent and Trademark Office, which is a continuation of U.S. application Ser. No. 16/074,211 filed on Jul. 31, 2018 (issued as U.S. Pat. No. 11,154,723 on Oct. 26, 2021), which in turn is a national stage entry under 35 USC § 371 of international application No. PCT/KR2017/005059 filed on May 16, 2017, and claim priority to Korean patent application Nos. 10-2016-0155918 filed on Nov. 22, 2016, and 10-2017-0059923 filled on May 15, 2017. The disclosures of the above-mentioned applications are incorporated by reference herein in their entirety.

### BACKGROUND

### 1. Field

The present disclosure relates to a transcranial stimulator, and more particularly, to a transcranial stimulator used for the purpose of rehabilitation of various neurodegenerative diseases based on a photobiomodulation mechanism.

### 2. Description of the Related Art

Recently, a solution for treating mental illness is being actively developed in advanced countries. For example, Aducanumab, which is an Alzheimer's treatment ingredient that has been conditionally approved by the US food and drug administration (FDA), has been introduced, but there are still controversies about its effectiveness, and concerns about safety are also a big deal.

In addition, a brain disease treating method for removing amyloid beta aggregates existing in brain cells through a transcranial stimulation technology is being studied.

The transcranial stimulation technology is a rehabilitation treatment technology that prevents, treats, and manages neurodegenerative disease such as Alzheimer's and Parkinson's by supplying specific energy into the skull.

The known transcranial stimulation technology has been implemented in a medical device type designed to stimulate the brain in a non-invasive manner or a minimally invasive manner, and details thereof are exemplified in the table illustrated in FIG. 13.

Referring to FIG. 13, a medical device for stimulating the brain through a transcranial focused ultrasound neuromodulation is first being developed. The medical device is a system that prevents brain disease by irradiating only the brain lesion with external ultrasound and is not an implantable medical device and is designed to form a focus of the ultrasound inside the brain, and thus, energy density is concentrated in a focused region. Accordingly, when the exact brain lesion site is not irradiated, there is a high risk of damage to other brain tissues, and there is a possibility that therapeutic effects are not achieved.

Next, a transcranial magnetic stimulation (TMS) method, which stimulates the brain by generating an external magnetic field, was introduced. This treatment method may not set a certain region of the brain as a treatment target and stimulates the brain as a whole. Accordingly, a brain tissue unrelated to the brain lesion is stimulated resulting in more stimulation than necessary, and there is a high possibility that a number of side effects, such as scalp pain, dizziness, headache, facial convulsions, drowsiness, and cognitive changes, are accompanied.

In addition to the non-invasive method, an invasive treatment technology for stimulating the brain by inserting a device into a human body to supply electrical energy is under study. One example is vagal nerve stimulation (VNS), which physically stimulates the brain by applying an electric current to the brain by inserting a vagal nerve device. This technology requires a professional surgery to insert an electrode wire by making an incision in the neck, and there is a limitation in that it is impossible to accurately target the brain lesion site.

In another example, a study on a deep brain stimulation (DBS) technology has been conducted. This technology has a concept in which brain disease is treated by implanting an electrode deeply at the base of the brain lesion site to apply a pulse from the outside, and requires high-risk electrode insertion. In addition, because a basic standard for an implantable medical device is applied, the cost of procedure is very high, and the use is limited because a high level of expertise is required.

As described above, the known transcranial stimulator may have a fatal risk when energy is delivered incorrectly, thereby being developed as an in-hospital-only medical device that may be treated by a specialist as a whole.

In addition, stimulation delivered to the brain produces various side effects such as vomiting, dizziness, and nausea, and therapeutic effects of brain diseases are not clearly verified yet.

### SUMMARY

The present disclosure provides a device for treating neurodegenerative disease based on photobiomodulation for transferring light energy under conditions effective for treating various neurodegenerative diseases or brain diseases to the brain of a human or animal.

A transcranial stimulator according to an embodiment of the present disclosure includes a main body formed in a shape that covers part or all of a head surrounding a skull, a light emission module configured in the main body to transmit light energy to a brain, and a controller configured to control an operation of the light emission module, wherein the light emission module includes a first optical device emitting light in a wavelength band of 630 nm to 680 nm toward the skull and a second optical device emitting light in a wavelength band of 780 nm to 990 nm toward the skull.

According to an embodiment of the present disclosure, the first optical device may dispersively emit light onto a wider area than the second optical device, and the second optical device may intensively emit light onto a narrower area than the first optical device.

According to an embodiment of the present disclosure, light emitted by the first optical device may be more effective in treatment of cell regeneration, and light emitted by the second optical device may be more effective in treatment for promoting or improving a blood flow in a cerebral blood vessel.

According to an embodiment of the present disclosure, the controller may control the light emission module to perform simultaneously or alternately a first emission operation for emitting light throughout a bottom of the skull and a second emission operation for allowing light to deeply penetrate into the skull, and the first emission operation may emit light in a relatively low wavelength range compared to the second emission operation.

According to an embodiment of the present disclosure, the controller may adjust an output value of the light emission module such that a temperature of a scalp does not exceed a preset normal range.

According to an embodiment of the present disclosure, the light emission module may include at least one optical device assembly in which at least one second optical device is arranged in the center and a plurality of first optical devices are arranged radially and symmetrical around the at least one second optical device.

According to an embodiment of the present disclosure, the first optical device may include a first low-power light source that emits light in a wavelength band of 630 nm to 680 nm, and the second optical device may include a second low-power light source that emits light in a wavelength band of 780 nm to 860 nm and a third low-power light source that emits light in a wavelength band of 890 nm to 990 nm to derive a therapeutic effect different from a therapeutic effect of the second low-power light source.

According to an embodiment of the present disclosure, the light emission module may include at least one optical device assembly in which at least one third low-power light source is arranged in the center, a plurality of second low-power light sources are arranged radially and symmetrical round the at least one third low-power light source, and a plurality of first low-power light source is arranged around a region where the plurality of second low-power light sources are arranged.

According to an embodiment of the present disclosure, the light emission module may include a frontal lobe light emission module arranged to face a frontal lobe of the brain to transfer light energy to the frontal lobe, a temporal lobe light emission module arranged to face a temporal lobe of the brain to transfer light energy to the temporal lobe, and an occipital lobe light emission module arranged to face an occipital lobe of the brain to transfer light energy to the occipital lobe.

According to an embodiment of the present disclosure, the light emission module may include a cervical spine light emission module arranged to face a cervical spine connected to the skull to transfer light energy to the brain through the cervical spine.

According to an embodiment of the present disclosure, the transcranial stimulator may further include a nasal cavity light emission applicator connected to the main body to transfer light energy to the brain by emitting light through a nasal cavity.

According to an embodiment of the present disclosure, the transcranial stimulator may further include a brainwave detection diagnosis module configured to diagnose a change in brainwave before and after light emission by analyzing a brainwave neural bio-signal collected according to light emission of the light emission module.

An embodiment of the present disclosure provides a transcranial stimulator, to which a photobiomodulation mechanism is applied, to prevent, treat, and manage neurodegenerative disease or mental illness.

One embodiment of the present disclosure provides a transcranial stimulator that reduces side effects of the body by minimizing direct physical stimulation to the brain.

One embodiment of the present disclosure provides a home transcranial stimulator that may be expanded to a solution for treating brain diseases in daily life by breaking the limitations of specialized hospital medical devices.

An embodiment of the present disclosure provides a transcranial stimulator that may target a light emission site in preparation for a major treatment of a neurodegenerative disease by exhibiting a clinically prescribed optimal light spec.

An embodiment of the present disclosure provides a transcranial stimulator that induces an optimal therapeutic effect by penetrating light in a specific wavelength band with low light loss.

An embodiment of the present disclosure provides a transcranial stimulator in which light sources are appropriately arranged for each brain region.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a view of a laser patch treatment set according to one embodiment of the present invention;

FIG. 2 is a view illustrating a patch bandage of the laser patch treatment set according to one embodiment of the present invention;

FIG. 3 is a perspective view of a laser patch according to one embodiment of the present invention;

FIG. 4 is a cross-sectional view illustrating components of the laser patch shown in FIG;

FIG. 5 is a flowchart illustrating a low level laser (LLL) treatment method according to one embodiment of the present invention;

FIG. 6 is a view of a laser patch according to one modified example of the present invention;

FIG. 7 is a view of a laser patch according to another modified example of the present invention;

FIG. 8 is a view of a laser patch treatment set according to another embodiment of the present invention;

FIG. 9 is a view illustrating a patch bandage of the laser patch treatment set according to another embodiment of the present invention;

FIG. 10 is a perspective view of a laser patch according to another embodiment of the present invention;

FIG. 11 is a perspective view illustrating the laser patch shown in FIG. 10 when viewed from the other side;

FIG. 12 is a flowchart illustrating a laser treatment method using the laser patch according to another embodiment of the present invention;

FIG. 13 illustrates concept of the known transcranial stimulation technique;

FIGS. 14A and 14B are perspective views of a transcranial stimulator according to an embodiment of the present disclosure;

FIGS. 15A and 15B illustrate concept of an effect of light irradiated by a light emission module according to an embodiment of the present disclosure;

FIG. 16 is an example view illustrating an emission form of a light emission module according to an embodiment of the present disclosure;

FIG. 17 is a cross-sectional view of a partial region of a transcranial stimulator including one optical device, according to an embodiment of the present disclosure;

FIGS. 18 and 19 are example views illustrating a first embodiment of an optical device assembly according to the present disclosure;

FIG. 20 is an example view illustrating a second embodiment of the optical device assembly according to the present disclosure;

FIGS. 21A and 21B are views illustrating a transcranial stimulator, in which a light emission module for each brain region is arranged, according to an embodiment of the present disclosure;

FIG. 22 is a perspective view of a nasal light emission applicator according to an embodiment of the present disclosure; and

FIG. 23 is a structural diagram of a brainwave detection diagnostic system according to an embodiment of the present disclosure.

### DETAILED DESCRIPTION

Hereinafter, embodiments of the present disclosure will be described in detail with reference to the accompanying drawings such that those skilled in the art may easily carry out the present disclosure. However, the present disclosure may be embodied in various different forms and is not limited to the embodiments described herein. In addition, in order to clearly illustrate the present disclosure in the drawings, parts irrelevant to the descriptions are omitted, and similar reference numerals are attached to similar parts throughout the specification.

Throughout the specification, when a portion is “connected” to another portion, this includes not only a case of being “directly connected” but also a case of being “electrically connected” with another component therebetween. In addition, when a portion “includes” a certain component, this means that other components may be further included, rather than excluding other components, unless otherwise stated.

Hereinafter, embodiments of the present disclosure will be described in detail with reference to the accompanying drawings.

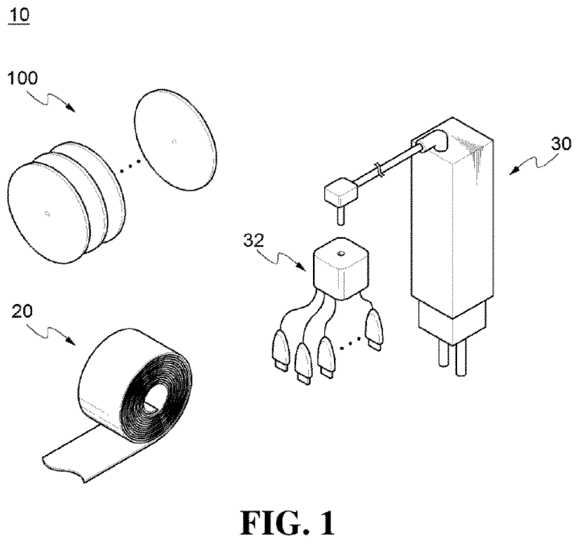

A laser patch and a laser patch treatment set including the same according to one embodiment of the present invention will be described in detail with reference to FIGS. 1 to 12.

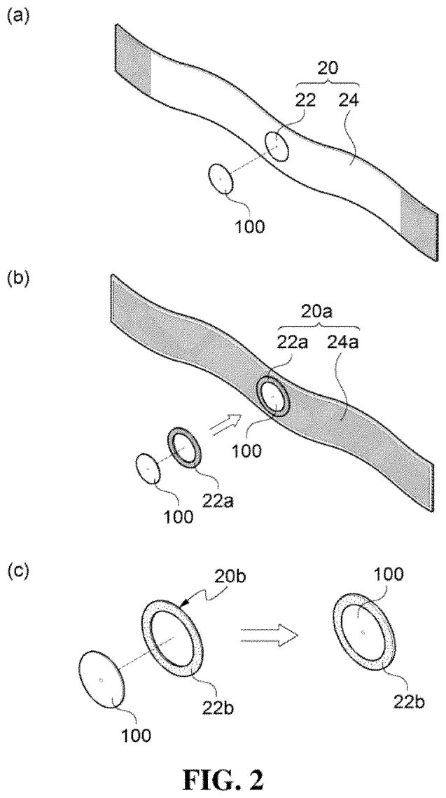

FIG. 1 is a view of the laser patch treatment set according to one embodiment of the present invention, and FIG. 2 is a view illustrating a patch bandage of the laser patch treatment set according to one embodiment of the present invention.

Referring to FIGS. 1 and 2, a laser patch treatment set 10 according to one embodiment of the present invention may include a patch bandage 20, a charger 30, a laser patch 100, and a packaging box (not shown) for accommodating and storing the components.

The laser patch 100 may be an apparatus which emits a relatively low level laser, that is, a low level laser (LLL) toward skin. As an example, the laser patch 100 may emit an LLL of about 80 mW or lower. The laser patch 100 may have a relatively small thin plate shape. The laser patch 100 may have a variety of shapes of planes such as a disc-shaped plane, a quadrangular plane, an oval plane, and the like. A plurality of such laser patches 100 are provided in one packaging box such that a user may use a variety of combinations of the plurality of laser patches 100.

The patch bandage 20 may allow the user to fix the laser patch 100 to a desired area on the user's skin. As shown in FIG. 2(a), the patch bandage 20 according to one embodiment of the present invention may include a fixing portion 22 for fixing one laser patch 100 and a bandage portion 24 connected to the fixing portion 22. The bandage portion 24 may dispose the laser patch 100 on the desired area and fix the laser patch 100 to a human body. In this case, the fixing portion 22 is provided as an opening formed in the bandage portion 24 such that the user may insert the laser patch 100 into the fixing portion 22 and then put the bandage portion 24 around the user's body to use.

As shown in FIG. 2(b), a patch bandage 20a according to another embodiment of the present invention may include a fixing portion 22a which has Velcro attached on one surface as form to be fitted onto the laser patch 100 and a bandage portion 24a with Velcro detachably attached to the Velcro of the fixing portion 22a. In this case, the user may attach the fixing portion 22a to the bandage portion 24a at a desired position corresponding to the desired area so as to use the laser patch 100, with which the fixing portion 22a is combined.

As shown in FIG. 2(c), the patch bandage 20b according to still another embodiment of the present invention includes a fixing portion 22b which is used while fitted onto the laser patch 100. Also, an adhesive material capable of adhering to or being in close contact with and being fixed to skin may be applied to one surface of the fixing portion 22b. In this case, the user may closely attach the laser patch 100 to the patch bandage 20b and then may attach the patch bandage 20b to a desired area to use.

The charger 30 may be for charging the laser patch 100. The charger 30 may include a charging gender 32 which is separate from or integrated with the charger 30 so as to charge a plurality of laser patches 100 at the same time.

Subsequently, the laser patch according to one embodiment of the present invention will be described in detail. Here, a redundant description on the above-described laser patch 100 may be omitted or simplified.

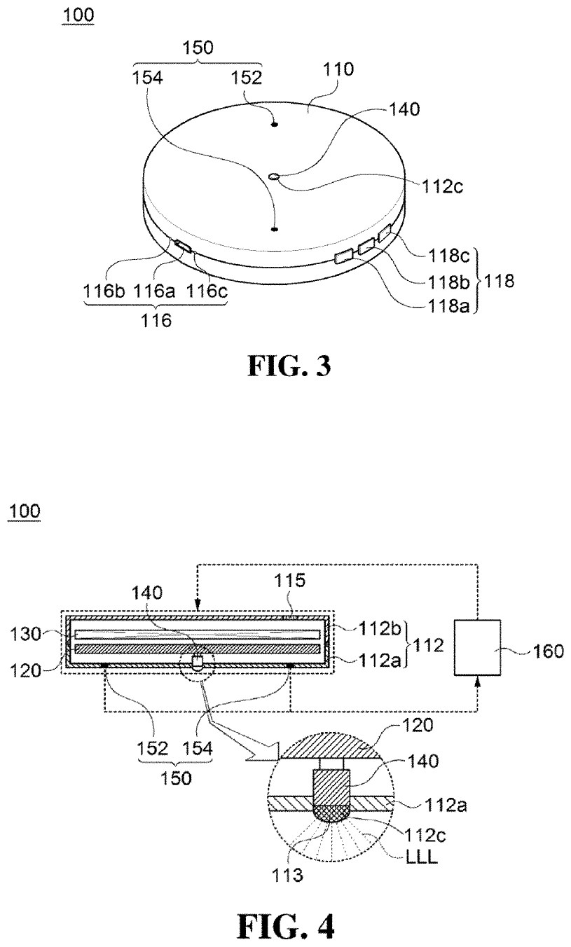

FIG. 3 is a perspective view of the laser patch according to one embodiment of the present invention, and FIG. 4 is a cross-sectional view illustrating components of the laser patch shown in FIG. 3.

Referring to FIGS. 3 and 4, the laser patch 100 according to one embodiment of the present invention may include a patch body 110, a circuit board 120, a built-in battery 130, a laser device 140, a sensing device 150, and a controller 160.

The patch body 110 may have a thin plate shape overall and may provide an internal space for protecting, arranging, and supporting the components 120, 130, 140, 150, and 160. As an example, the patch body 110 may include a first body 112a and a second body 112b which are assembled with each other to form a thin plate-shaped body 112. The first body 112a may have one surface which comes into close contact with a human body during treatment, and the second body 112b may have the other surface which is exposed during treatment. A through hole 112c for transmitting an LLL may be provided in the one surface of the first body 112a. The second body 112b may include an operational light 115 which allows the user to recognize emission of the LLL or operation of the laser patch 100.

Meanwhile, a lens 113 for diffusion of the LLL may be further provided in the through hole 112c of the first body 112a. As an example, the lens 113 may be configured to reduce linearity of light of a general laser so as to emit the light toward a wider area in a human body. For this, a convex lens may be used as the lens 113 such that the LLL which passes through the through hole 112c may be transmitted through the lens 113 and then may diffuse so as to penetrate a relatively wide human body area. In this case, it may be suitable for advantageous purposes, that is, pain relief, lymph node stimulation, edema relief, skin stimulation, and the like to dispersedly emit LLLs toward a relatively wide area.

As another example, the lens 113 may be provided to maintain or strengthen linearity of the LLL. For this, a concave lens or a general transmission plate may be used as the lens 113 such that the LLL which passes through the through hole 112c may be transmitted through the lens 113 and then may converge or be maintained so as to penetrate a relatively narrow human body area. In this case, it may suitable for purposes of blood flow improvement, blood flow strengthening, cell activation, and the like which need intensive emission of the LLL toward a relatively narrow area.

A charging portion 116 may be provided in the patch body 110 for connecting the charger 30 to charge the built-in battery 130. The charging portion 116 may include a charging terminal 116a and a first charge light 116b and a second charge light 116c for allowing recognition of whether or not charging is completed or a residual battery amount. The first charge light 116b may include a green light emitting diode (LED) which indicates completion of charging, and the second charge light 116c may include a red LED which indicates charging.

Also, the patch body 110 may include a variety of control buttons 118 for setting a treatment condition of the laser patch 100. For example, the control buttons 118 may include an on/off button 118a for turning on/off the LLL device 140, a laser level adjusting button 118b for adjusting a laser output intensity of the LLL device 140, and a timer button 118c for setting an operation time of the laser patch 100. The user may set treatment conditions of the laser patch 100 by setting the above-described control buttons 118.

The circuit board 120 may be disposed inside the patch body 110. The circuit board 120 may be disposed to be adjacent to and face the first body 112a in comparison to the built-in battery 130. The circuit board 120 may include at least one printed circuit board (PCB).

The built-in battery 130 may be a battery able to be repeatedly charged. The built-in battery 130 may be charged by the charger 30 and may supply power for laser emission of the LLL device 140 as internal power. A lithium polymer battery may be used as the built-in battery 130 for reducing the risk of explosion and ignition. The built-in battery 130 may allow the laser patch 100 to operate due to its own power without additional external power.

The LLL device 140 may be provided on the circuit board 120 to emit an LLL to the outside through the through hole 112c. A general LLL is known as having a variety of effects such as pain relief, blood flow improvement, blood flow strengthening, cell activation, lymph node stimulation, edema relief, skin stimulation, and the like, and these effects are provided by LLLs having a certain wavelength range. For example, when LLLs are emitted toward a human body for 20 minutes or more as an output of 30 mW or more at a wavelength range of about 630 nm to 680 nm, effects of pain relief, lymph node stimulation, edema relief, cell activation, and the like are relatively high. When LLLs are emitted toward a human body for 20 minutes or more as an output of 30 mW or more at a wavelength range of about 780 nm to 990 nm, effects of blood flow improvement, blood flow strengthening, cell activation, and the like are relatively high. Accordingly, as the LLL device 140, devices having a variety of wavelength ranges may be used according to the above-described purposes.

Here, the above-described LLL device 140 releases much heat outward when generating a laser. The heat of the LLL device 140 may shorten the lifetime of a device, deteriorate the reliability and safety of a product, and particularly, may cause a patient to be burned during treatment. Accordingly, a means capable of effectively releasing heat of the LLL device 140 is necessary. As the means, an additional heat sink (not shown) may be disposed near the LLL device 140. However, in this case, a thickness of the laser patch 100 increases due to at least a degree of protrusion from the circuit board 120. However, the laser patch 100 according to one embodiment of the present invention may have a maximally thin thickness for user convenience. That is, the laser patch further protrude toward skin as the thickness thereof increases such that a sense of difference or discomfort must increase when the laser patch 100 comes into close contact with the skin and aesthetics thereof are not good.

In order to address the above-described part, the laser patch 100 according to one embodiment of the present invention includes the patch body 110 formed of a material having relatively high thermal conductivity so as to be used as a heat sink for discharging the heat of the LLL device 140 outward or as a heat transfer plate for transferring heat to a human body during treatment. For this, the patch body 110 may include stainless steel, aluminum, copper, and a variety of other metal materials entirely or may include a multilayer structure having a biomaterial in which a skin contact surface is formed of a plastic material which satisfies biocompatibility and an inside thereof is formed of a metal material. In this case, in the laser patch 100, the patch body 110 is partially or entirely heated due to heat generated by the LLL device 140 such that appropriate heat of the patch body 110 is transferred to the human body so as to perform thermal therapy. That is, the above-described laser patch 100 may be configured to perform basic LLL treatment and the thermal therapy using the own heat of the patch body 110 at the same time.

The sensing device 150 may be provided for safety during treatment of the laser patch 100 and provided to appropriately control treatment. For example, the sensing device 150 may include a contact sensor 152 and a temperature sensor 154. The contact sensor 152 may sense whether one surface of the laser patch 100 comes into contact with the user's skin while the laser patch 100 is used. For this, as the contact sensor 152, a pressure sensor, an impedance sensor, a magnetic sensor, a capacitance sensor, and the like may be used. Otherwise, the contact sensor 152 may be a sensor which senses a contact with skin by using a physical or mechanical means. For example, as the contact sensor 152, a physical sensor which is provided with an ordinary protruding pin, and when coming into close contact with the skin, is inserted thereinto and senses whether contact is present.

The temperature sensor 154 may prevent a user from getting burned during treatment because the laser patch 100 is heated by heat generated by the LLL device 140. As described above, the patch body 110 according to one embodiment of the present invention may be formed of a material having relatively high thermal conductivity and may be configured to use heat of the LLL device 140 for thermal therapy. However, in this case, since a burn may occur due to excessive heat generation of the laser patch 110, a safety technique for preventing the occurrence of burns may be necessary. For this, the patch body 110 may include at least one temperature sensor 154 to sense a temperature of the patch body 110. As an example, the temperature sensor 154 may be provided in the first body 112a of the patch body 110 to sense a temperature of the first body 112a which comes into direct contact with a human body during treatment.

The controller 160 may receive a sensing signal from the sensing device 150 and may control the laser patch 100. For example, the controller 160 may receive a contact sensing signal from the contact sensor 152, may turn on the LLL device 140 when the patch body 110 comes into contact with a human body during treatment, and may turn off the LLL device 140 when the patch body 110 does not come into contact with the human body. Also, the controller 160 may receive a temperature sensing signal from the temperature sensor 154, may turn off the LLL device 140 when a temperature of the patch body 110 deviates from a preset temperature, and may turn on the LLL device 140 when the temperature of the patch body 110 satisfies a preset temperature. Here, the preset temperature range may be a temperature range appropriate for thermal therapy. For example, the preset temperature range may be from about 40° C. to 55° C. During treatment, when the patch body 110 is less than about 40° C., a thermal therapy effect may be extremely slight. On the other hand, when the patch body 110 is more than about 55° C., a user may get burned.

Subsequently, an LLL self-treatment method using the above-described laser patch treatment set will be described in detail.

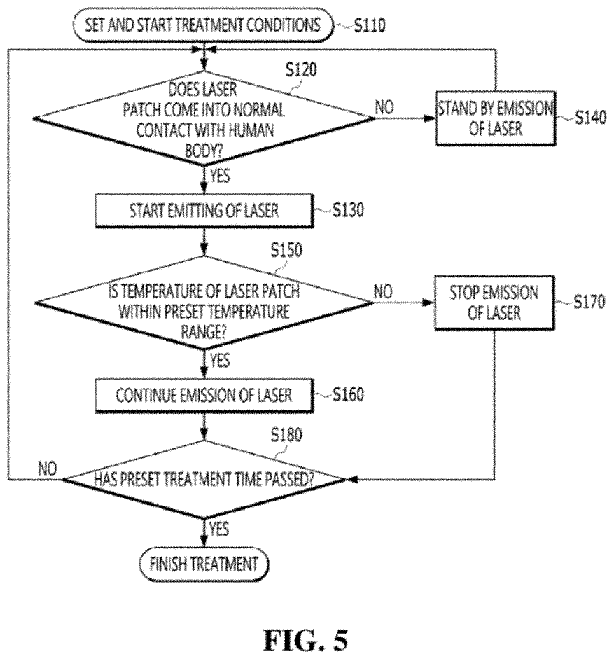

FIG. 5 is a flowchart illustrating an LLL treatment method according to one embodiment of the present invention. Referring to FIGS. 3 to 5, first, treatment conditions may be set and started (S110). In more detail, a user may set an output time and intensity adjustment of an LLL of the laser patch 100 and whether to perform an operation of turning on (On) by manipulating the control buttons 118 of the laser patch 100. Setting values of the above treatment conditions may differ for each of purposes such as pain relief, blood flow improvement, blood flow strengthening, skin regeneration, cell activation, lipolysis, cell stimulation, and the like.

As an example, when a purpose is pain relief, the output of the LLL is set to 40 mW by manipulating the laser level adjusting button 118b, set to 30 minutes by manipulating the timer button 118c, and the laser patch 100 may be turned on by manipulating the on/off button 118a in the above condition. Also, the laser patch 100 may be selected and fixed to the patch bandage 20 which is suitable for skin tissue of a target and then may be attached to an area to be treated.

When the laser patch 100 is attached to the area to be treated, it may be determined whether the laser patch 100 comes into normal contact with a human body (S120). In more detail, the controller 160 may receive a contact sensing signal from the contact sensor 152 of the sensing device 150 and may determine whether one surface of the first body 112a of the laser patch 100 is normally attached to the area to be treated. Here, when it is determined that the patch body 110 comes into normal contact with the area to be treated, the controller 160 may control the circuit board 120 such that the LLL device 140 may start emitting an LLL by using own power of the built-in battery 140 (S130). On the other hand, when it is determined that the patch body 110 does not come into contact with the area to be treated, the controller 160 may cause the LLL emission of the LLL device 14 to be on standby (S140).

That is, the LLL emission of the LLL device 140 may be performed only when the laser patch 100 comes into normal contact with the area to be treated and otherwise may always be on standby in an off state. Accordingly, the laser patch 100 is allowed to emit the LLL only when coming into normal contact with the area to be treated such that it is possible to prevent a negligent accident such as emitting an LLL toward eyes due to the negligence of a user.

Also, when emission of the LLL is performed, it may be determined whether a temperature of the laser patch 100 is within a preset temperature range (S150). In more detail, the controller 160 may receive a temperature sensing signal from the temperature sensor 154 of the sensing device 150 and may determine whether a temperature of the patch body 110 of the laser patch 100 is within the preset temperature range. Here, when the temperature of the patch body 110 is within the preset temperature range, the controller 160 may allow the LLL device 140 to continue the emission of the LLL (S160). On the other hand, when the temperature of the patch body 110 deviates from the preset temperature range, the controller 160 may control the circuit board 120 to stop the LLL emission of the LLL device 140 (S170).

That is, as described above, since the laser patch 100 according to one embodiment of the present invention uses heat of the LLL device 140 as heat for thermal therapy, when the temperature of the patch body 110 excessively increases, a problem such as burns may occur. Accordingly, the above-described laser patch 100 is allowed to emit the LLL only when the temperature does not deviate from a normal temperature range such that safety of treatment may increase.

During a process of performing the above-described laser treatment, it may be determined that a preset treatment time has passed (S180). For example, when 30 minutes which is set in the above-described operation S110 have not passed, the controller 160 may return to the operation S120 and may repeatedly perform the above-described operations S120 to S170. On the other hand, when 30 minutes which is set in the above-described operation S110 have passed, the LLL device 140 is turned off and then an alarm or external display such as an off light of the operational light 115 may be indicated to allow the user to recognize it.

As described above, the laser patch treatment set 10 according to one embodiment of the present invention includes the laser patches 100 which emit LLLs and the patch bandage 20 capable of fixing the laser patch 100 to the area to be treated. Each of the laser patches 100 may have a relatively small diameter and may have a thin plate shape. In this case, the laser patches 100 may be combined to be selectively arranged in local areas such as a painful area, blood apertures, lymph nodes, and the like such that a variety of self-treatments such as pain relief, blood flow improvement, lymph node stimulation, edema relief, skin stimulation, and the like may be performed. Accordingly, according to the embodiments of the present invention, a laser patch and a laser patch treatment set including the same includes relatively small plate-shaped laser patches to perform treatment in a local area such that the patches may be appropriately combined to fit a necessary treatment type and a desired local area by the user.

Also, according to the embodiments of the present invention, a laser patch and a laser patch treatment set including the same may perform treatments for pain relief, blood flow improvement, blood flow strengthening, skin regeneration, cell activation, lipolysis, cell stimulation, and the like without skin troubles such as a skin rash, an allergy, a burn, and the like which are problems caused by an adhesive and a patch in comparison to general patches or pain relief patches.

Also, according to the embodiments of the present invention, a laser patch and a laser patch treatment set including the same may allow a user to perform self-treatment with the same effect at no additional cost for a long time and prevent resource waste to be eco-friendly in comparison to general patches or pain relief patches.

Subsequently, laser patches according to modified examples of the present invention will be described. Here, a description which overlaps with that of the laser patch 100 according to one embodiment of the present invention may be omitted or simplified.

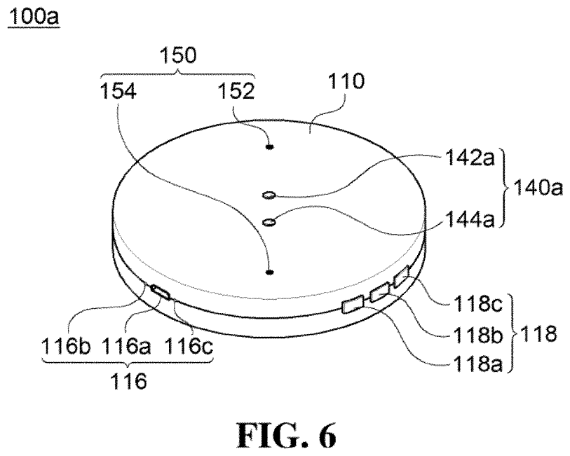

FIG. 6 is a view of a laser patch according to one modified example of the present invention. Referring to FIG. 6, a laser patch 100a according to one modified example of the present invention may include the patch body 110, the circuit board 120, the built-in battery 130, an LLL device 140a, the sensing device 150, and the controller 160. The LLL device 140a may include a first device 142a and a second device 144a having different laser wavelength values. As an example, the first device 142a may include an LLL device having a wavelength range of about 630 nm to 680 nm and an output intensity of about 80 mW or less. The first device 142a may be used for purposes such as pain relief, lymph node stimulation, edema relief, cell activation, and the like. On the other hand, the second device 144a may include an LLL device having a wavelength range of about 780 nm to 990 nm and an output intensity of about 80 mW or less. The second device 142a may be used for purposes such as blood flow improvement, blood flow strengthening, cell activation, and the like.

The numbers and arrangements of the first and second devices 142a and 144a may be variously changed. A user may select and control the first and second devices 142a and 144a by manipulating the control buttons 118. For example, when the laser patch 100a is used for pain relief, the user may set the control buttons 118 to turn on only the first device 142a and use the laser patch 100a. Otherwise, when the laser patch 100a is used for blood flow improvement, the user may set the control buttons 118 to turn on only the second device 144a and use the laser patch 100a. When the user intends to use the laser patch 100a for both pain relief and blood flow improvement at the same time, the user may turn on both the first and second devices 142a and 144a and then use the laser patch 100a.

The laser patch 100a having the above-described structure may emit, as one laser patch 100a, lasers having different wavelengths so as to perform laser treatment having two wavelengths by a single laser patch 100a in comparison to the laser patch 100 according to one embodiment of the present invention.

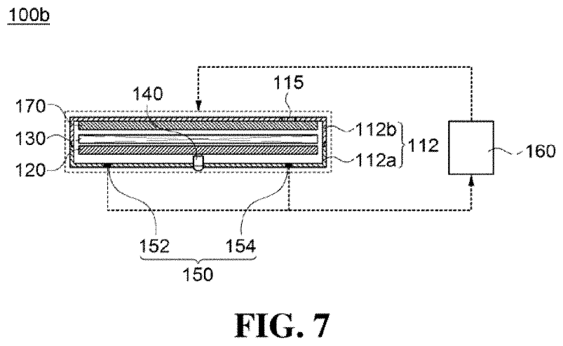

FIG. 7 is a view of a laser patch according to another modified example of the present invention. Referring to FIG. 7, a laser patch 100b according to another modified example of the present invention may include the patch body 110, the circuit board 120, the built-in battery 130, the LLL device 140a, the sensing device 150, the controller 160, and an oscillator 170. The oscillator 170 may be provided to further add purposes such as blood flow improvement and the like by transferring micro oscillations to an area to be treated during treatment. As the oscillator, an ultrasonic oscillator, an electromagnetic motor oscillator, and the like may be used.

The laser patch 100b having the above-described structure may simultaneously or separately perform LLL treatment and micro oscillation treatment as a single laser patch 100b.

Hereinafter, a laser patch and a laser patch treatment set including the same according to another embodiment of the present invention will be described in detail with reference to the attached drawings. Here, a description which overlaps with those of the above-described laser patches 100, 100a, and 100b may be omitted or simplified.

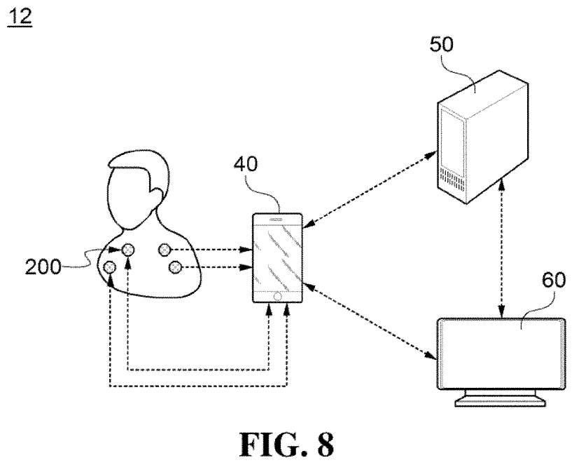

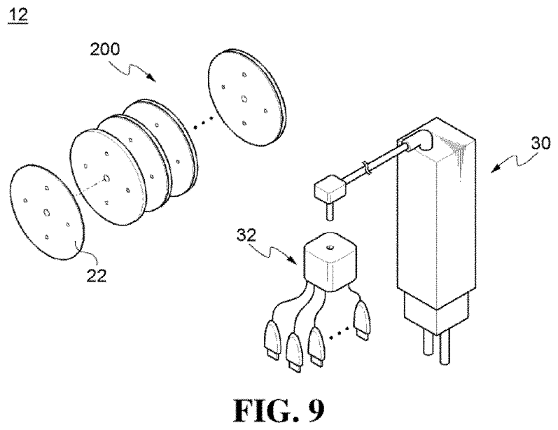

FIG. 8 is a view of a laser patch treatment set according to another embodiment of the present invention, and FIG. 9 is a view illustrating a patch bandage of the laser patch treatment set according to another embodiment of the present invention.

Referring to FIGS. 8 and 9, a laser patch treatment set 12 according to another embodiment of the present invention may include a patch bandage 22, the charger 30, a user terminal 40, a web server 50, a web client 60, a laser patch 200, and a packaging box (not shown) for accommodating and storing the components.

The laser patch 200 is an apparatus which emits an LLL toward skin and may have a relatively small thin plate shape. A user may perform self-treatment of Korean medicine treatments such as acupuncture, moxa cautery treatment, and the like by variously combining a plurality of such laser patches 200.

The patch bandage 22 may allow the user to fix the laser patch 200 to a desired area on the user's skin. As shown in FIG. 9, the patch bandage 22 according to another embodiment of the present invention may be provided as an adhesive bandage which is attached to one surface of the laser patch 200. For this, one surface of the patch bandage 22 which comes into contact with skin may be provided as a form to which an adhesive material which is harmless to a human body is applied, and the other surface thereof may be provided as a form which is attached to a laser emission surface of the laser patch 200. The patch bandage 22 may include a plurality of holes formed therein so as not to interfere with a path for lasers emitted by the laser patch 200. During treatment, the user may attach the patch bandage 22 to each of the laser patches 200 and then may allow the patch bandage 22 to come into close contact with desired skin tissue to use.

The charger 30 may be for charging the laser patch 100. The charger 30 may include the charging gender 32 which is separate from or integrated with the charger 30 so as to charge a plurality of such laser patches 200 at the same time.

The user terminal 40 may be a device on which an application for allowing the user to check and control a method of using the laser patch 200, a history, and the like is installed. As the user terminal 40, a variety of portable terminals such as a smart phone and a tablet PC may be used. The user terminal 40 may wirelessly communicate with the laser patch 200 through Bluetooth, Zigbee, radio frequency (RF), and the like. Otherwise, the user terminal 40 may communicate with the laser patch 200 through wires using a USB connection portion and the like. The application may display a variety of pieces of information on LLL treatment such that the user may use the laser patch 200 on the basis of the information.

The web server 50 may include a database including treatment information data on Korean medicine treatments such as basic acupuncture and moxa cautery treatment and may check and manage the database. Also, basic information which is input by the user through the user terminal 40 may be recognized, and the user may be guided with treatment information in the database which is appropriate for the user and the method of using the laser patch 200 through preset conditions and a calculation program.

The web client 60 may receive a variety of pieces of treatment information and the history and data of the laser patch 200 from the user terminal 40 and the web server 50.

Subsequently, the laser patch according to another embodiment of the present invention will be described in detail. Here, a description which overlaps with those of the variously formed laser patches 100, 100a, and 100b may be omitted or simplified.

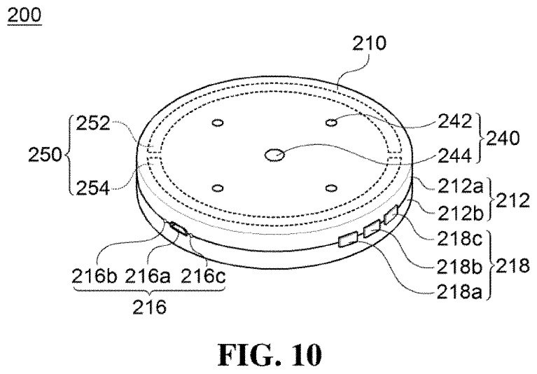

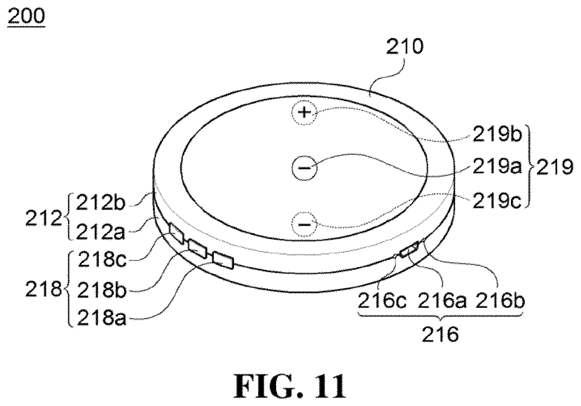

FIG. 10 is a perspective view of a laser patch according to another embodiment of the present invention, and FIG. 11 is a perspective view illustrating the laser patch shown in FIG. 10 when viewed from the other side.

Referring to FIGS. 10 and 11, the laser patch 200 according to another embodiment of the present invention may include a patch body 210, a circuit board (not shown), a built-in battery (not shown), a laser device 240, a sensing device 250, and a controller (not shown). The circuit board, the built-in battery, the controller, and the like may be equal or similar to the components of the above-described laser patches 100, 100a, and 100b.

The patch body 210 may include a first body 212a and a second body 212b which are assembled with each other to form a thin plate-shaped body 212. The first body 212a may have one surface which comes into close contact with a human body during treatment, and the second body 212b may have the other surface which is exposed during treatment. A charging portion 216 may be provided in the patch body 210 for connecting the charger 30 to charge the built-in battery. The charging portion 216 may include a charging terminal 216a and a first charge light 216b and a second charge light 216c for allowing recognition of whether or not charging is completed or a residual battery amount.

The patch body 210 may include a variety of control buttons 218 for setting a treatment condition of the laser patch 200. For example, the control buttons 218 may include the on/off button 218a for turning on/off the LLL device 240, a laser level adjusting button 218b for adjusting a laser output intensity of the LLL device 240, and a timer button 218c for setting an operation time of the laser patch 200. Also, the patch body 210 may further include an oscillator control button 219 for setting and operating an oscillator (not shown) provided in the laser patch 200. For example, the oscillator control button 219 may include an on/off button 219a for turning on/off the oscillator, selecting an oscillation mode, or the like, an increase button 219b for increasing the intensity of the oscillator, and a decrease button 219c for decreasing the intensity of the oscillator. The user may set treatment conditions of the laser patch 200 by setting the above-described control buttons 218 and 219.

The LLL device 240 may include a first device 242 and a second device 244 having different laser wavelength values. As an example, the first device 242 may include an LLL device having a wavelength range of about 630 nm to 680 nm and an output intensity of about 80 mW or less. The first device 242 may be used for purposes such as pain relief, lymph node stimulation, edema relief, cell activation, and the like. On the other hand, the second device 244 may include an LLL device having a wavelength range of about 780 nm to 990 nm and an output intensity of about 80 mW or less. The second device 242 may be used for purposes such as blood flow improvement, blood flow strengthening, cell activation, and the like.

The numbers and arrangements of the first and second devices 242 and 244 may be variously changed. For example, at least one second device 244 may be disposed in a central area of one surface of the patch body 210 and a plurality of such first devices 242 may be arranged in a peripheral area of the one surface of the patch body 210. In the LLL device 240 having the above-described structure, the second device 244 is located above a blood vessel of a user to increase or improve blood flow in a particular blood vessel and the other first devices 242 emit LLLs toward a relatively wide area to relief pain.

The sensing device 250 may be provided for safety during treatment of the laser patch 200 and provided to appropriately control treatment. For example, the sensing device 250 may include a contact sensor 252 and a temperature sensor 254. The contact sensor 252 may sense whether one surface of the laser patch 200 comes into contact with skin of the user when the laser patch 200 is used, and the temperature sensor 254 may prevent the user from getting burned during treatment because the laser patch 200 is heated by heat generated by the LLL device 240.

The controller may receive a sensing signal from the sensing device 250 and may control the laser patch 200. For example, the controller may receive a contact sensing signal from the contact sensor 252, may turn on the LLL device 240 when the patch body 210 comes into contact with a human body during treatment, and may turn off the LLL device 240 when the patch body 210 does not come into contact with the human body. Also, the controller may receive a temperature sensing signal from the temperature sensor 254, may turn off the LLL device 240 when a temperature of the patch body 210 deviates from a preset temperature, and may turn on the LLL device 240 when the temperature of the patch body 210 satisfies a preset temperature.

Subsequently, an LLL treatment process using the above-described laser patch according to another embodiment of the present invention will be described in detail. Here, a description which overlaps with those of the laser patches according to the above-described variety of embodiments may be omitted or simplified.

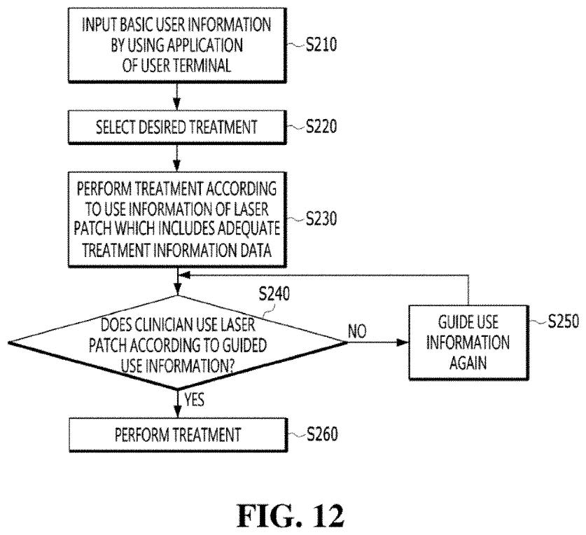

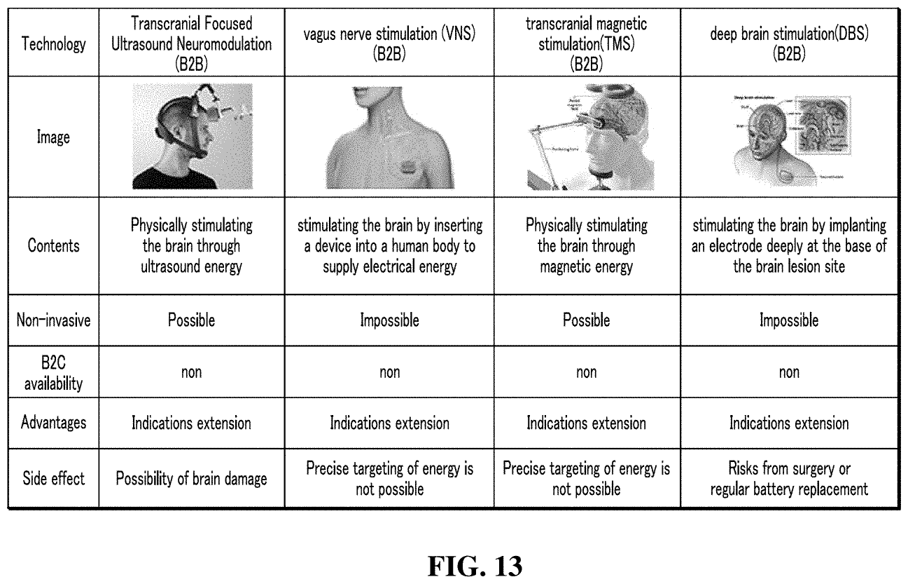

FIG. 12 is a flowchart illustrating a laser treatment method using the laser patch according to another embodiment of the present invention. Referring to FIGS. 8 to 12, basic user information may be input by using an application of the user terminal 40 (S210). For example, a user may download a related application through the user terminal 40 and may input basic user items, for example, gender, age, weight, disease to be treated, and the like.

After inputting of the basic user information, the user may select a desired treatment (S220). In more detail, the user may input a treatment item and a position to be treated through the application. For example, when the user inputs pain treatment among pain treatment and blood flow improvement which are classified as major items, treatment areas such as arms, legs, shoulders, waist, and the like which are classified as intermediate items appear. When the shoulders among them are input, a right side, a left side, another detailed painful area, and the like may appear. The user may input the desired treatment and the most appropriate treatment item through the detailed classifications.

Treatment may be performed according to use information of the laser patch including appropriate treatment information data (S230). In more detail, when the user selects the desired treatment, the web server 50 may transmit the appropriate treatment information data from information basically stored in a database, to the user terminal 40. The user may perform the treatment by sequentially attaching the laser patches 200 to an area to be treated according to a method or process of using the laser patches 200 which is shown through the user terminal 40.

Particularly, the treatment information data may include a treatment guide having at least one purposes of pain relief, blood flow improvement, cell activation, cell regeneration, lymph node stimulation, and edema relief such that a treatment sequence and positions are set with respect to the laser patches through the treatment guide selected by previously input information of the user and the user is allowed to sequentially locate the laser patches in the set positions of the user according to the set treatment sequence and positions. Accordingly, the user may perform treatments as Korean medicine acupuncture or a moxa cautery process while sequentially attaching the laser patches 200 to particular positions such that an ordinary person may easily perform specialized Korean medicine self-treatment.

It may be determined whether a clinician uses the laser patches according to the guided use information in the above-described treatment process (S240). For example, the controller (not shown) may determine whether the user performs the treatment according to the treatment process guided through the user terminal 40 on the basis of whether the laser patch 200 comes into contact with a human body when the user attaches the laser patch 200 to the human body which is transmitted from the sensing device 250. When the user performs the treatment by using the laser patches 200 according to the guided treatment process, it is determined to be normal use such that normal treatment may be performed (S260). Here, the normal treatment may include an LLL treatment which has been described above with reference to FIG. 5. On the other hand, when the user does not perform the treatment by using the laser patches 200 according to the guided treatment process, the user may be notified of it and the use information may be guided again so as to perform appropriate treatment (S250).

As described above, the laser patch 200 and the laser patch treatment set 12 including the same according to another embodiment of the present invention may include the laser patches 200 which perform LLL treatments, the user terminal 40 on which the application which communicates with the laser patches 200 and guides the method and process of using the laser patches 200 is installed, and the web server 50 connected to the user terminal 40 such that an ordinary user may be guided with a desired treatment procedure by using the application and may perform Korean medicine self-treatment by using the laser patches 200. Accordingly, according to the embodiments of the present invention, there are provided a laser patch and a laser patch treatment set including the same which are configured to connect to a user terminal on which an application which guides a user of the laser patches with an accurate procedure for acupuncture or moxa cautery treatment, which is a Korean medicine treatment, is loaded such that an ordinary user who has no or lacks Korean medicine treatment knowledge may simply and accurately perform Korean medicine self-treatment.

The laser patch 200 according to an embodiment of the present disclosure may be deformed in shape depending on the field of application, and may typically serve as a transcranial stimulator.

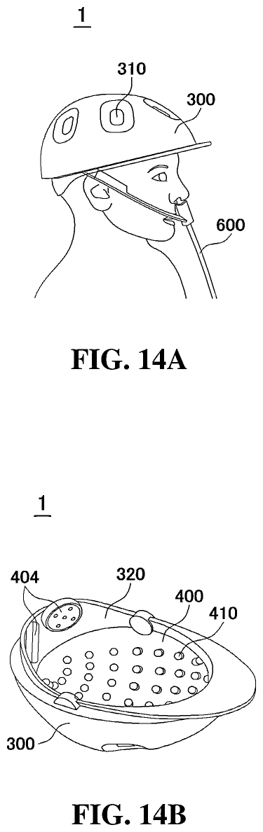

Hereinafter, a transcranial stimulator based on photobiomodulation mechanism according to one embodiment of the present invention will be described in detail with reference to FIGS. 14 to 23.

FIGS. 14A and 14B are perspective views of a transcranial stimulator according to an embodiment of the present disclosure.

A transcranial stimulator 1 according to the present disclosure includes a main body 300 formed in a shape to cover a part or all of the head. FIG. 14A is a view illustrating an exterior of the main body 300 in a state in which a user wears the transcranial stimulator 1, and FIG. 14B is a view illustrating the inside of the main body 300.

According to one embodiment, as illustrated in FIG. 14A, the main body 1 is preferably formed in a helmet shape such that a user may wear the main body 1 easily, but the shape implemented as a structure that covers or wraps the head is not limited in particular to the wearable shape.

The transcranial stimulator 1 according to an embodiment may include a heat dissipation module 310 for dissipating heat, which is generated as light is irradiated, to the outside. For example, as illustrated in FIG. 14A, the heat dissipation module 310 may include at least one heat dissipation member arranged along an exterior of the main body 10.

The transcranial stimulator 1 according to an embodiment may include a support band 320 for supporting a user's head. The support band 320 may cover scalp of a skull such that the transcranial stimulator 1 is fixed to the user's head. Accordingly, since the transcranial stimulator 1 is closely attached to the forehead along a circumference thereof, the main body 300 may be supported, and the scalp may be protected.

The transcranial stimulator according to the present disclosure includes a light emission module 400 configured inside the main body 300 to transfer light energy to the brain and a controller 500 for controlling an operation of the light emission module 400. That is, light of the light emission module 400 emitted by the controller 500 causes a photobiomodulation mechanism to achieve prevention and treatment effect of neurodegenerative disease or mental illness.

Here, photobiomodulation (PBM) refers to a mechanism in which adenosine triphosphate (ATP) is activated by light irradiated to the body. According to a PBM technology, balanced active oxygen, which induces cell repair and healing, is generated, and in this process, a nerve chain blocked by nitric oxide (NO) is pierced, and the nitric oxide (NO) returns to a body system. As described above, the nitric oxide (NO) generated according to light absorption is involved in transfer of nutrients and oxygen to cells such that cell regeneration is active and helps to expand blood vessels and improve blood circulation.

The transcranial stimulator 1 according to the present disclosure uses a PBM technology and does not cause side effects such as vomiting, nausea, and dizziness, unlike the known transcranial stimulation techniques and uses a non-invasive method that does not require a high-risk procedure, and thus, it is possible to be applied as a home solution for prevention and treatment of brain disease.

According to an embodiment, the light emission module 400 may include one more optical devices 410. For example, as illustrated in FIG. 14B, the optical devices 410 may be arranged to face the skull in a preset pattern along an inner surface of the main body 300 so as to cover the entire brain area.

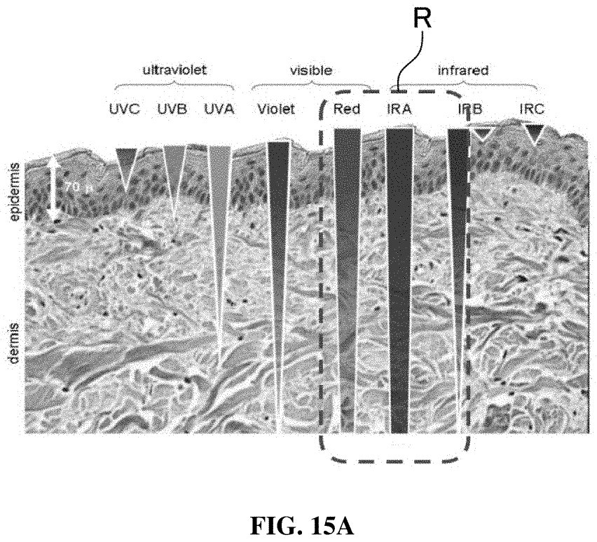

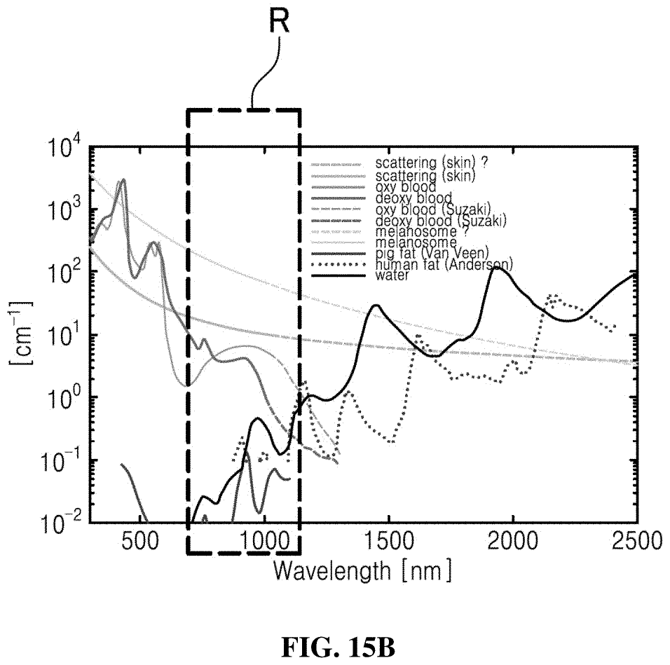

According to an embodiment, the optical device 410 may be designed to output light having a wavelength in a preset range with high light transmittance. As illustrated in FIG. 15A, it can be seen that a part of a visible ray region and light in a wavelength band R of a near-infrared region penetrate deeply into the skin. In addition, as illustrated in FIG. 15B, it can be seen that the light of in the wavelength band R exhibits the lowest light loss when penetrating into the body, thereby providing optimal light transmittance. The optical device 410 according to the present disclosure outputs light having a preset wavelength within the wavelength band R, and thus, a significant therapeutic effect may be obtained, compared to the known PBM technology using light in a low permeability region.

In one embodiment, the optical device 410 may be designed to emit light having at least one wavelength in a wavelength band of 630 nm to 990 nm.

In one embodiment, the optical device 410 may be designed to emit light having at least one wavelength in a wavelength band of 630 nm to 860 nm.

In one embodiment, the optical device 410 may be designed to emit light in a wavelength band of 630 nm to 680 nm and light in a wavelength band of 780 nm to 990 nm.

In one embodiment, the optical device 410 may be designed to emit light in a wavelength band of 630 nm to 680 nm and light in a wavelength band of 780 nm to 860 nm.

In one embodiment, the optical device 410 may be designed to emit light in a wavelength band of 630 nm to 680 nm, light in a wavelength band of 780 nm to 860 nm, and light in a wavelength band of 890 nm to 990 nm.

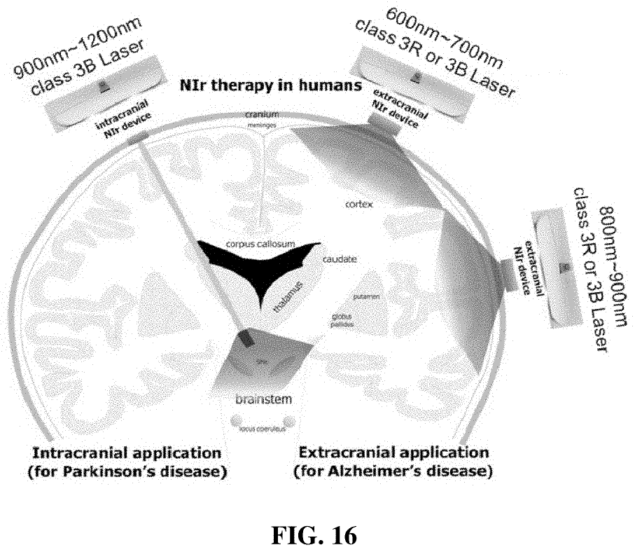

According to an embodiment, as illustrated in FIG. 16, the transcranial stimulator 1 may be designed to target both a bottom tissue of the brain and a deep tissue of the brain. Specifically, the controller 500 may control the light emission module 400 such that a first emission operation for irradiating light throughout the bottom of the skull and a second emission operation for allowing light to penetrate deeply into the skull are simultaneously or alternately performed. Meanwhile, the wavelength band illustrated in FIG. 16 is for explanation, and the present disclosure is not limited thereto.

According to one embodiment, the first emission operation may be set to emit light having a relatively low wavelength range compared to the second emission operation. For example, treatment and prevention of a brain bottom tissue, the first emission operation may be set to emit light in a complex wavelength condition having a wavelength band of 630 nm to 700 nm and a wavelength band of 800 nm to 900 nm. In addition, for treatment and prevention of a deep tissue of the brain, the second emission operation may be set to emit light in a complex wavelength condition having a wavelength band of 830 nm to 860 nm and a wavelength band of 900 nm to 990 nm.

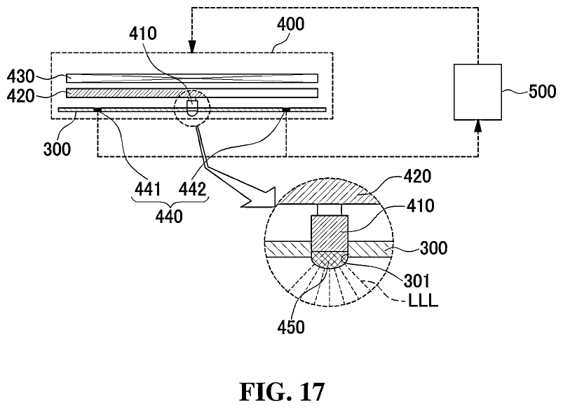

Hereinafter, an embodiment of an internal configuration of the transcranial stimulator 1 will be described with reference to FIG. 17.

FIG. 17 is a cross-sectional view of a partial region of a transcranial stimulator including one optical device, according to an embodiment of the present disclosure. In FIG. 17, the main body 300 of the partial region is illustrated as a shape of a plate, but this is for illustration and the entire main body 300 may be manufactured with a predetermined curvature to cover a part or all of the head.

Referring to FIG. 17, the light emission module 400 according to an embodiment may be configured between an outer body of the main body 300 and an inner body facing scalp and include a circuit board 420, a built-in battery 430, and an optical device 410.

The circuit board 420 according to an embodiment may include at least one printed circuit board (PCB) and may be configured by at least one flexible printed circuit board (FPCB) in order to correspond to a curvature of the head. The circuit board 420 is preferably arranged adjacent to an inner body according to mounting of the optical device 410, but a position of the arrangement does not limit the present disclosure.

According to the embodiment, the built-in battery 430 may be manufactured as a battery that may be repeatedly charged. The built-in battery 430 may be charged by a charger (not illustrated) configured in the main body 300 and may supply power for light emission of the optical device 410 as an internal power source. As described above, the transcranial stimulator 1 includes the built-in battery 430 so as to operate with its own power without external power, and thus, portability is greatly improved.

According to one embodiment, it is preferable that the built-in battery 430 is designed to use the transcranial stimulator 1 in a predetermined number of times (at least two times or more) after charging. In this case, it is possible to prevent a phenomenon in which light is output inconsistently during use and to improve convenience of a procedure.

According to an embodiment, the optical device 410 may be arranged inside the transcranial stimulator 1 to emit light to the brain through a penetration hole 301. That is, the optical device 410 may include at least one light source exposed to the outside through the penetration hole 301 configured in the main body 300.

According to an embodiment, a predetermined lens 450 supplementing light emitted by the optical device 410 may be configured in the penetration hole 301. For example, the convex lens 450 for diffusing light emitted to the outside to a wider area may be configured. In this case, the light passing through the convex lens 450 spreads widely and penetrates the brain, and thus, the light is advantageously used for the purpose of treating a relatively large area of the brain.

In another example, a concave lens 450 or a general penetration plate 450 for maintaining and enhancing straightness of light emitted to the outside may be configured. In this case, the light passing through the concave lens 450 intensively penetrates into a target region, and thus, the light is advantageously used for the purpose of intensively treating a relatively narrow area of the brain.

According to an embodiment, the type of light source included in the optical device 410 is not limited, but a low-level laser (LLL) may be a representative example. Low-power laser in a specific wavelength band penetrated into a human body or an animal body exerts various effects, such as pain relief, cell activation, blood flow improvement, blood flow promotion, lymph node stimulation, edema relief, and skin irritation. Therefore, it is preferable to arrange a low-power laser in a wavelength band, which exerts a relatively high therapeutic effect for each specific part of the brain, in the main body 300.

According to an embodiment, a light source may be designed to have a preset output intensity in order for light energy to constantly reach the bottom and a deep portion of the brain. For example, one light source may be designed to have an error range of 20 percent of 100 mW, and the transcranial stimulator 1 may include an adjustment device (not illustrated) that may adjust output intensity of the light source.

According to an embodiment, operation time of a light source may be kept constant in order to obtain a stable treatment effect. That is, the controller 500 may have a preset time for the light source to operate and emit light when the transcranial stimulator 1 is used. In addition, in order to maximize a therapeutic effect, the total energy emitted by the transcranial stimulator 1 has to be constantly maintained above a preset level. To this end, the number of light sources included in the transcranial stimulator 1 may be determined based on the preset output intensity and operation time of the light source.

According to one embodiment, in order to maximize the therapeutic effect by generating light energy of a certain level or more and to prevent a user from being damages due to a lifespan of the light emission module 400 and heat, the main body 300 may be made of a material that dissipates heat effectively. That is, the main body 300 may be made of a material with high thermal conductivity to serve as a heat dissipation member for dissipating heat to the outside or a heat transfer member for transferring heat into the body during use.

For example, the main body 300 is made of stainless steel, aluminum, copper, or various other metal materials or may also be designed as a multi-layered structure which is made of a heterogeneous material and of which skin contact surface is made of a predetermined material satisfying biocompatibility and of which inner side is made of a metal material. In this case, part or all of the main body 300 is heated due to the heat generated by the optical device 410, and the resultant heat is transferred to the body to exert an effect of thermal treatment.

According to one embodiment, the transcranial stimulator 1 may include a predetermined safety device 440 for safety of a user. For example, at least one of a touch sensor 441 and a temperature sensor 442 may be provided in one region of the main body 300. In addition, each of the touch sensor 441 and the temperature sensor 442 may be arranged in plurality on the main body 300 at a preset separation distance.

The touch sensor 441 may detect whether an inner surface of the main body 300 is in contact with a user's scalp or reaches a predetermined distance or less when the transcranial stimulator 1 is used. Here, the touch sensor 441 may include a pressure sensor, an impedance sensor, a magnetic sensor, a capacitive sensor, and other physical and mechanical sensors but is not limited thereto.

The temperature sensor 442 may detect in advance whether the heat generated by the optical device 400 reaches a certain level or more to prevent a user from being burnt or damaged and detect a temperature of the main body 300 or a temperature of a scalp

The controller 500 may control an operation of the transcranial stimulator 1 based on detection signals received from the touch sensor 441 and the temperature sensor 442. For example, the controller 500 operates the light emission module 400 when it is determined that the scalp is in contact with the main body 300 or the scalp is located below a preset distance according to a signal of the touch sensor 441, while the controller 500 stops the light emission module 400 when the scalp is not in contact with the main body 300 or moves away from the main body 300.

In addition, the controller 500 may operate the light emission module 400 only when a temperature received from the temperature sensor 442 is in a preset normal range. That is, when the transcranial stimulator 1 is used and the temperature is out of the normal range, the controller 500 may control the light emission module 400 to operate or stop, and a normal range may be set to a range in which an appropriate thermal treatment is performed and the scalp is not damaged. For example, a normal range may be between 40 degrees Celsius and 55 degrees Celsius, but preferably it does not exceed preferably 41 degrees Celsius.

According to an embodiment, when the temperature is out of the normal range, the controller 500 may control the light emission module 400 to maintain a normal temperature. That is, the controller 500 may adjust an output value of the light emission module 400 such that a temperature of the scalp does not exceed a preset normal range. Accordingly, a user may stably use the transcranial stimulator 1 without intermediate change.

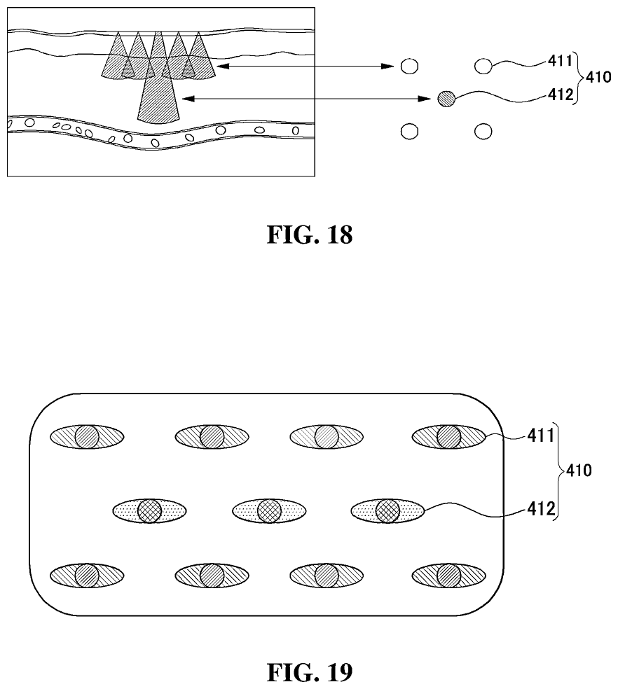

Hereinafter, a configuration and an arrangement of an optical device according to the present disclosure will be described with reference to FIGS. 18 to 20.

The light emission module 400 according to an embodiment may include first optical devices 411 and a second optical devices 412.

The first optical devices 411 according to an embodiment may each include at least one light source (for example, a low-power laser) emitting light in a wavelength band of 630 nm to 680 nm toward a skull. Efficiency of light in the wavelength band of 630 nm to 680 nm penetrating into a brain has been proved, and the wavelength band has been identified as one wavelength band that exhibits an optimal photobiomodulation mechanism.

The second optical devices 412 according to an embodiment may each include at least one light source (for example, a low-power laser) emitting light in a wavelength band of 780 nm to 990 nm toward a skull. Efficiency of light in the wavelength band of 780 nm to 990 nm penetrating into a brain has been proved, and the wavelength band has been identified as one wavelength band that exhibits an optimal photobiomodulation mechanism.

Referring to FIG. 18, the first optical devices 411 according to an example embodiment may be designed to dispersively emit light to a relatively wide emission area compared to the second optical devices 412. That is, the first optical devices 411 may be arranged at a preset interval in one region of the main body 300 so as to target the bottom of the brain as a whole.

Meanwhile, the second optical devices 412 according to an example embodiment may be designed to intensively emit light to a relatively narrow emission area compared to the first optical devices 411. That is, the second optical devices 412 may be configured in the main body 300 to intensively target a deep portion of the brain and may be preferably arranged in a region corresponding to a region, which is relatively easily penetrated, within the skull.

According to an embodiment, a convex lens may be configured in the penetration hole 301, to which a light source of the first optical device 411 is exposed, to increase an effect of diffusion, or a concave lens is configured in the penetration hole 301, in which a light source of the second optical device 412 is exposed, to increase straightness of light.

According to an embodiment, light emitted by the first optical devices 411 and the second optical devices 412 may exert various therapeutic effects according to photobiomodulation, but may be designed to intensively exert relatively different treatment mechanisms.

Specifically, light in a wavelength band of 630 nm to 680 nm was confirmed as an optimal photobiomodulation condition for activating cell regeneration by activation of bioenergy (ATP). Accordingly, the light emitted by the first optical devices 411 is more effective in treatment of cell regeneration.

In addition, light in a wavelength band of 780 nm to 990 nm was confirmed as an optimal photobiomodulation condition related to cerebrovascular blood flow treatment. Accordingly, the light emitted by the second optical devices 412 is more effective in treatment for promoting or improving a blood flow in a cerebral blood vessel.

According to an embodiment, in order to maximize an effect of cell regeneration, a wavelength band of the light emitted by the first optical devices 411 may be formed in a narrower range. For example, light having a wavelength corresponding to a wavelength of about 650 nm may be emitted by the first optical devices 411.

According to an embodiment, in order to maximize an effect of improving a blood flow in a brain blood vessel, a wavelength band of the light emitted by the second optical devices 412 may be formed in a narrower range. For example, light in a wavelength band of 780 nm to 860 nm may be emitted by the second optical devices 412, and light having a wavelength corresponding to a wavelength of about 830 nm is preferably emitted.

According to an embodiment, the light emission module 400 may include at least one optical device assembly in which the optical devices 410 are arranged in a predetermined shape. Accordingly, it is possible to accurately target the bottom and a deep portion of the brain depending on parts of the brain to maximize efficiency of treatment and prevention of brain diseases.

As illustrated in FIG. 18, an optical device assembly may be implemented in the form in which one of the second optical devices 412 is arranged in the center and four first optical devices 411 are arranged radially and symmetrically around the second optical device 412. In this case, a therapeutic effect on a deep tissue of the brain at the position where the optical device assembly is arranged may be intensively controlled by the second optical devices 412, and a therapeutic effect on relatively wide brain base tissues around the position may be controlled by the first optical devices 411.

In addition, an arrangement shape may be variously changed depending on the number of optical devices 400 included in the optical device assembly. For example, as illustrated in FIG. 19, an optical device assembly may be implemented in the form in which three second optical devices 412 are arranged in the center and eight first optical devices 411 are arranged radially and symmetrically around the three second optical devices 412.

That is, an optical device assembly may be designed in the form of the first embodiment in which one or more second optical devices 412 are arranged in the center and a plurality of first optical devices 411 may be arranged radially and symmetrically around the one or more second optical devices 412. That is, an optical device assembly may be appropriately arranged as the number of the first optical devices 411 and the second optical devices 412 is determined according to a target region and a target area.

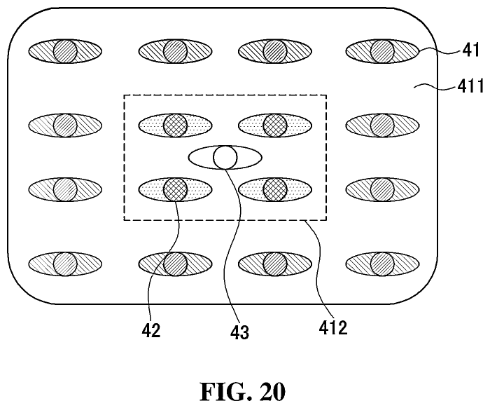

Referring to FIG. 20, an optical device assembly may be implemented in the form in which light sources classified in three different wavelength bands are arranged in a preset arrangement.

According to a related embodiment, the first optical device 411 include first low-power light sources 41 emitting light in a wavelength band of 630 nm to 680 nm, and the second optical device 412 may include second low-power light sources 42 that emit light in a wavelength band of 780 nm to 860 nm and a third low-power light source 43 that emits light in a wavelength band of 890 nm to 990 nm. In this case, the second low-power light sources 42 and the third low-power light source 43 may derive different therapeutic effects and preferably derive various therapeutic effects according to photobiomodulation but may be designed to centrally exert different therapeutic mechanisms.

As illustrated in FIG. 20, the optical device assembly may be designed in the form in which one or more third low-power light sources 43 are arranged in the center, and a plurality of second low-power light sources 42 are arranged radially and symmetrically around the one or more third low-power light sources 43, and a plurality of first low-power light sources 41 are arranged around the region where the plurality of second low-power light sources 42 are arranged. That is, the optical device assembly may be appropriately arranged as the number of the first low-power light sources 41, the second low-power light sources 42, and the third low-power light sources 43 is determined according to a target region and a target area.

Meanwhile, although the first and second embodiments of the light emission assembly are introduced in the present specification, the number of light sources and an arrangement may change depending on a treatment purpose, a treatment target, treatment site and area of the transcranial stimulator 1, and forms for solving the same technical problem should be interpreted as being included in the scope of the present disclosure.

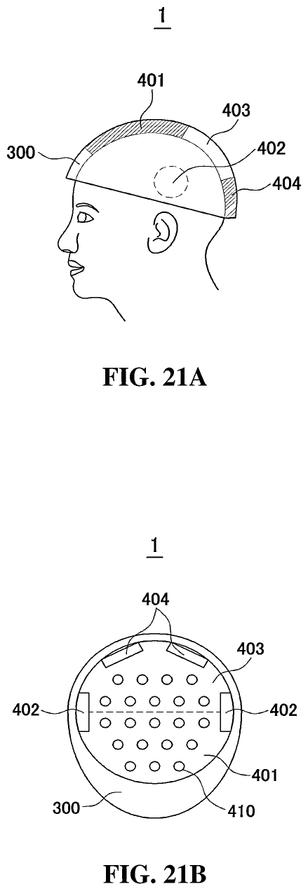

Hereinafter, an embodiment of the light emission module 400 divided for each brain region will be described with reference to FIGS. 21A and 21B.

FIGS. 21A and 21B are views illustrating a transcranial stimulator in which a light emission module for each brain region is arranged, according to an embodiment of the present disclosure.

According to an embodiment, characteristics of a skull and tissues are different for each part of the brain, and thus, it is advantageous for treatment for the transcranial stimulator 1 to have an appropriate light source arrangement for performing accurate targeting for each part.

In this regard, the transcranial stimulator 1 according to an embodiment may be designed in a form in which the light emission module 400 is divided for each part. Specifically, referring to FIGS. 21A and 21B, the light emission module 400 may include a frontal lobe light emission module 401, temporal lobe light emission modules 402, and an occipital lobe light emission module 403.

The frontal lobe light emission module 401 according to an embodiment is arranged to face a frontal lobe of a brain and serves to transfer light energy to the frontal lobe. The frontal lobe light emission module 401 may include one or more first optical devices 411 to target the entire bottom of the frontal lobe and may include one or more second optical devices 412 to allow light to penetrate deeply into the frontal lobe.

According to an embodiment, the frontal lobe light emission module 401 may include at least one optical device assembly according to the first embodiment. For example, the frontal lobe light emission module 401 may be designed in a form in which at least two (preferably, three) second optical devices 412 are arranged in the center and a plurality of first optical devices 411 are arranged radially and symmetrical around the second light device 412.

According to an embodiment, the prefrontal lobe light emission module 401 may include at least one optical device assembly according to the second embodiment. Alternatively, the prefrontal lobe light emission module 401 may be designed in a combined form including at least one optical device assembly according to the first embodiment and at least one optical device assembly according to the second embodiment.

The temporal lobe light emission modules 402 according to an embodiment are arranged on the left and right temporal lobes of the brain to face each other and serve to transfer light energy to the temporal lobes. The temporal lobe light emission modules 402 may each include one or more first optical devices 411 to target the entire bottom of the temporal lobes and may each include one or more second optical devices 412 to allow light to penetrate deeply into the temporal lobes.

According to an embodiment, the temporal lobe light emission modules 402 may each include at least one of optical device assembly according to the first embodiment or the optical device assembly according to the second embodiment. For example, in consideration of areas of the temporal lobes, the temporal lobe light emission modules 402 on the left and right may also be designed in a compact form in which one second optical device 412 is arranged in the center and a plurality of first optical devices 411 are arranged radially and symmetrical around the one second light device 412.

The occipital lobe light emission module 403 according to an embodiment is arranged to face the occipital lobe of the brain and serves to transfer light energy to the occipital lobe. The occipital lobe light emission module 403 may include one or more first optical devices 411 to target the entire bottom of the occipital lobe and include one or more second optical devices 412 to allow light to penetrate deeply into the occipital lobe.

According to an embodiment, the occipital lobe light emission module 403 may include at least one optical device assembly according to the first embodiment. Alternatively, the occipital lobe light emission module 403 may include at least one optical device assembly according to the second embodiment. Alternatively, the occipital lobe light emission module 403 may be designed in a combined form including at least one optical device assembly according to the first embodiment and at least one optical device assembly according to the second embodiment.

The light emission module 400 according to an embodiment may further include cervical spine light emission modules 404. The cervical spine light emission modules 404 are arranged to face the cervical spine connected to the skull and serve to transfer light energy to the brain through the cervical spine.

According to an embodiment, the cervical spine light emission modules 404 may each include at least one first optical device 411 to target the bottom of the brain connected to the cervical spine and include at least one second optical device 412 to allow light to penetrate deeply into the brain, which is connected to the cervical spine.

According to an embodiment, the cervical spine light emission modules 404 may each include at least one optical device assembly according to the first embodiment or at least one optical device assembly according to the second embodiment. For example, in consideration of an area and a shape of the cervical spine, a compact optical device assembly according to the first embodiment including one second optical device 412 may be arranged on the left and right of the central axis of the cervical spine to be separated from each other.

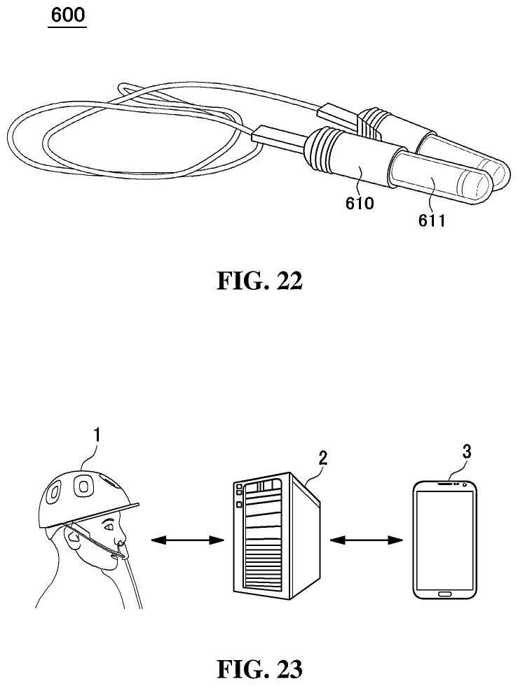

Meanwhile, the transcranial stimulator 1 according to an embodiment of the present disclosure may further include a predetermined optional device for more efficient light emission of brain tissue. In particular, a nasal light emission applicator 600 that transfers light energy to the brain by emitting light through a nasal cavity may be further configured, and an example embodiment thereof is illustrated in FIG. 22.

Referring to FIG. 22, the nasal light emission applicator 600 according to an embodiment may be designed as an insert-type structure that is connected to the main body 300 and inserted into the nasal cavity of a person or an animal. For example, the nasal light emission applicator 600 may include probes 610 that may be inserted into the left and right nasal cavities, and optical devices 611 may be provided at end portions of the probes.

According to an embodiment, the optical devices 611 may include both a first optical device and a second optical device or may include any one of the first and second optical devices, depending on purposes or depths of treatment. For example, when improvement or promotion of a blood flow is required more in cerebral blood vessels, the nasal light emission applicator 600, in which the second optical devices are respectively connected to the left and right probes 610, may be connected to the main body 300 to be used. Meanwhile, when treatment of cell regeneration is required more, the nasal light emission applicator 600, in which the first optical devices are connected to the left and right probes 610, may be used. Alternatively, when targeting of the bottom and a deep portion of the brain is required more, the nasal light emission applicator 600, in which the first optical device is connected to one of the probes 610 and the second optical device is connected to the other of the probes 610, may be used.

Hereinafter, an embodiment of brainwave detection diagnosis using the transcranial stimulator 1 will be briefly introduced with reference to FIG. 23.

FIG. 23 is a structural view of a brainwave detection diagnostic system according to an embodiment of the present disclosure.

Referring to FIG. 23, the brainwave detection diagnosis system may include a transcranial stimulator 1, a diagnosis server 2, and a display device 3.

According to an embodiment, the transcranial stimulator 1 may perform network communication and transmit brainwave neural bio-signals collected according to the use of the transcranial stimulator 1 to the diagnosis server 2.

The diagnosis server 2 according to an embodiment may include a memory (not illustrated) in which a program (or an application) for performing a brainwave detection diagnosis method using a transcranial stimulator (not illustrated) is stored and a processor (not illustrated) that executes the program described above.

The brainwave detection diagnosis method according to an embodiment may include a step of collecting brainwave neural bio-signals of a user from the transcranial stimulator 1 and building a database, a step of processing and analyzing the brainwave neural bio-signals to derive a result of a brainwave change before and after an optical procedure using the transcranial stimulator 1, and a step of providing the derived result of the brainwave change to the user through the diagnosis device 3.

According to an embodiment, a preset machine learning algorithm may be applied to a process of processing and analyzing a brainwave neural bio-signal. The machine learning algorithm may include a supervised learning method and an unsupervised learning method, and for example, the supervised learning method may include a decision tree, k-nearest neighbors (KNN), naive bayes, a support vector machine (SVM), random forest, a deep neural network (DNN), a convolutional neural network (CNN), a recurrent neural network (RNN), a long short term memory (LSTM), a generative adversarial network (GAN), and so on but is not limited thereto.

The display device 3 according to an embodiment serves to display the result of the brainwave change received from the diagnosis server 2 and total diagnosis information according thereto. The display device 3 may also be manufactured as an option configured with the transcranial stimulator 1 as a set or may also be implemented in a form of a general user terminal. In the latter case, a program (or an application) for providing a brainwave detection diagnosis service may be stored in the user terminal in conjunction with the transcranial stimulator 1.

Meanwhile, the transcranial stimulator 1 according to an embodiment may further include a brainwave detection diagnosis module (not illustrated), and the brainwave detection diagnosis module may be included in the main body 300 or may be connected to the main body 300 as a separate device. For example, when implemented as a separate device, the brainwave detection diagnosis module may replace a role of the display device 3 illustrating a diagnosis result.

In addition, when implemented as a separate device, the brainwave detection diagnosis module may include a memory (not illustrated) storing a program (or an application) for performing a brainwave detection diagnosis method using a transcranial stimulator (not illustrated) and a processor (not illustrated) that executes the program. That is, a role of the diagnosis server 2 may also be replaced, and in this case, regular updates of the program may be provided from the server providing the program.

The above description of the present disclosure is for illustration, and those skilled in the art to which the present disclosure pertains will understand that the description may be easily modified into other specific forms without changing the technical idea or essential features of the present disclosure. Therefore, it should be understood that the embodiments described above are illustrative in all respects and not restrictive. For example, each component described as a single type may also be implemented in a distributed manner, and likewise components described as distributed may also be implemented in a combined form.

The scope of the present disclosure is indicated by the following claims rather than the above detailed description, and all changes or modifications derived from the meaning and scope of the claims and their equivalent concepts should be interpreted as being included in the scope of the present disclosure.

What is claimed is:

1. A transcranial stimulator comprising:
a main body formed in a shape that covers part or all of a head surrounding a skull; a light emission module configured in the main body to transmit light energy to a brain; and
a controller configured to control an operation of the light emission module,
wherein the light emission module includes a first optical device emitting light in a wavelength band of 630 nm to 680 nm toward the skull and a second optical device emitting light in a wavelength band of 780 nm to 990 nm toward the skull.

2. The transcranial stimulator of claim 1, wherein
the first optical device dispersively emits light onto a wider area than the second optical device, and the second optical device intensively emits light onto a narrower area than the first optical device.

3. The transcranial stimulator of claim 1, wherein
light emitted by the first optical device is more effective in treatment of cell regeneration, and light emitted by the second optical device is more effective in treatment for promoting or improving a blood flow in a cerebral blood vessel.

4. The transcranial stimulator of claim 1, wherein
the controller controls the light emission module to perform simultaneously or alternately a first emission operation for emitting light throughout a bottom of the skull and a second emission operation for allowing light to deeply penetrate into the skull, and
the first emission operation emits light in a relatively low wavelength range compared to the second emission operation.

5. The transcranial stimulator of claim 1, wherein
the controller adjusts an output value of the light emission module such that a temperature of a scalp does not exceed a preset normal range.

6. The transcranial stimulator of claim 1, wherein
the light emission module includes at least one optical device assembly in which at least one second optical device is arranged in the center and a plurality of first optical devices are arranged radially and symmetrical around the at least one second optical device.

7. The transcranial stimulator of claim 1, wherein
the first optical device includes a first low-power light source that emits light in a wavelength band of 630 nm to 680 nm, and
the second optical device includes a second low-power light source that emits light in a wavelength band of 780 nm to 860 nm and a third low-power light source that emits light in a wavelength band of 890 nm to 990 nm to derive a therapeutic effect different from a therapeutic effect of the second low-power light source.

8. The transcranial stimulator of claim 7, wherein
the light emission module includes at least one optical device assembly in which at least one third low-power light source is arranged in the center, a plurality of second low-power light sources are arranged radially and symmetrical round the at least one third low-power light source, and a plurality of first low-power light source is arranged around a region where the plurality of second low-power light sources are arranged.

9. The transcranial stimulator of claim 1, wherein the light emission module comprises:
a frontal lobe light emission module arranged to face a frontal lobe of the brain to transfer light energy to the frontal lobe;
a temporal lobe light emission module arranged to face a temporal lobe of the brain to transfer light energy to the temporal lobe; and
an occipital lobe light emission module arranged to face an occipital lobe of the brain to transfer light energy to the occipital lobe.

10. The transcranial stimulator of claim 1, wherein
the light emission module includes a cervical spine light emission module arranged to face a cervical spine connected to the skull to transfer light energy to the brain through the cervical spine.

11. The transcranial stimulator of claim 1, further comprising:
a nasal cavity light emission applicator connected to the main body to transfer light energy to the brain by emitting light through a nasal cavity.

12. The transcranial stimulator of claim 1, further comprising:
a brainwave detection diagnosis module configured to diagnose a change in brainwave before and after light emission by analyzing a brainwave neural bio-signal collected according to light emission of the light emission module.
